(19) United States
(12) Patent Application Publication
(10) Pub. No.: US 2006/0271045 A1
(43) Pub. Date: Nov. 30, 2006

(54) SPINAL CROSS-CONNECTOR

(75) Inventors: Dennis Hubbard, Lancaster, MA (US); Donna Sommerich, Norton, MA (US)

Correspondence Address: NUTTER MCCLENNEN & FISH LLP, WORLD TRADE CENTER WEST, 155 SEAPORT BOULEVARD, BOSTON, MA, 02210-2604, US

(73) Assignee: DEPUY SPINE, INC., Raynham, MA (US)

(21) Appl. No.: 10/908,845

(22) Filed: May 27, 2005

(51) Int. Cl. A61F 2/30

(52) U.S. Cl. 606/61

(57) ABSTRACT

An implantable spinal cross-connector is provided for connecting one or more spinal fixation elements, and more preferably for connecting two spinal fixation rods that are implanted within a patient's spinal system. In general, the cross-connector includes a central portion having at least one connector member formed on a terminal end thereof and having first and second opposed jaws, at least one of which is selectively movable between a first, open position wherein the first and second jaws are positioned a distance apart from one another, and a second, closed position, wherein the first and second jaws are adapted to engage a spinal fixation element therebetween. The cross-connector also includes a cantilevered member that provides a feedback response when the spinal fixation rods are positioned within the cross-connector.

### BACKGROUND OF THE INVENTION

Fixation devices are used in spinal surgery to align and/or fix a desired relationship between adjacent vertebral bodies. Such devices typically include a spinal fixation element, such as a relatively rigid fixation rod, that is coupled to adjacent vertebrae by attaching the element to various anchoring devices, such as hooks, bolts, wires, or screws. Often two rods are disposed on opposite sides of the spinous process in a substantially parallel relationship. The fixation rods can have a predetermined contour that has been designed according to the properties of the target implantation site, and once installed, the rods hold the vertebrae in a desired spatial relationship, either until desired healing or spinal fusion has taken place, or for some longer period of time.

Spinal cross-connectors are often used in conjunction with spinal fixation devices to provide additional stability to the devices. For example, it has been found that when a pair of spinal rods is fastened in parallel on either side of the spinous process, the assembly can be significantly strengthened by using a cross-connector to bridge the pair of spinal rods. The connectors are typically in the form of a rod having a clamp formed on each end thereof for mating with a spinal rod.

Conventional spinal cross-connectors (and other such implanted devices) often include a receiving area into which a rod (or other such element) must be seated prior to securing the devices to one another. However, seating an implanted spinal device within a cross-connector can be challenging because of difficulty visualizing the connection. For example, portions of the fixation devices and/or anatomical features may obstruct a surgeon's view. As a result, a surgeon may be unsure if a cross-connector has been properly positioned. Moreover, since the cross-connector is often applied as the last step in a lengthy surgical procedure, ease of application is paramount.

Accordingly, there presently exists a need for a cross-connector device that facilitates secure and convenient mating with spinal fixation devices.

### BRIEF SUMMARY OF THE INVENTION

Described herein is an implantable spinal device that provides a feedback response when a spinal fixation device is seated therein. Such a feature improves the ease of use, rendering assembly of components during a surgical procedure more convenient and reliable.

In one embodiment, the device comprises a cross-connector including at least one connector member having a main body and first and second cooperating jaws that define a seating area therebetween. A cantilevered member extends into the seating area and is movable between a neutral and an expanded position in response to insertion of the spinal fixation element into the seating area. As the spinal fixation element enters the seating area, the cantilevered member provides a feedback response. The feedback response can be audible, tactile or a combination of an audible and a tactile feedback response.

In one aspect, the first and/or second jaw is unitary with the main body. Alternatively, only one of the jaws is unitary with the main body and the other jaw is formed from a separate component that is mated to the main body. The second jaw can be adapted to move between a first, open position and a second position effective to secure a spinal fixation element in the seating area. In another aspect, the device further includes a locking mechanism effective to move at least one of the first and second jaws between an open position and a closed position in which the jaws are effective to secure a spinal fixation element therebetween.

The cantilevered member can be separate from or unitary with the main body. In one aspect the cantilevered member is connected to the main body by a living hinge. The living hinge imparts flexibility to the cantilevered member allowing it to pivot between the neutral position and the expanded position.

In one embodiment there is provided a cross-connector having at least one connector member formed on a terminal end thereof. The connector member has first and second opposed jaws, at least one of which is movable between a first, open position in which the jaws are positioned a first distance apart from the other, and a second, closed position wherein the jaws are spaced so as to engage a spinal fixation element therebetween. The cross-connector further includes a cantilevered member positioned between the first and second jaws to define a receiving space between the cantilevered member and one of the first and second jaws having a dimension less than the first distance. In one aspect, a connector member is disposed on each terminal end of the cross-connector.

In another embodiment disclosed herein, an implantable system is provided. The system comprises a spinal fixation element including at least a portion thereof having a substantially cylindrical shape and a cross-connector including a main body with at least one jaw extending therefrom. The at least one jaw can define at least a portion of a seating area able to receive a spinal fixation element. The system further includes a cantilevered member extending from the main body and spaced apart from the at least one jaw by a distance less than a diameter of the spinal fixation element. The cantilevered member is adapted to deflect to enable the spinal fixation element to be inserted into the seating area and to return to its original position once the spinal fixation element is disposed in the seating area.

In yet another embodiment, a method of connecting to a spinal implant is provided. The method includes the steps of providing at least one connector member having a main body and first and second cooperating jaws defining a seating area. The seating area is adapted to receive a spinal fixation element therebetween. The method further includes positioning a spinal implant within the seating area such that a feedback response is generated in response to insertion of the spinal fixation element into the seating area. The feedback response can be tactile, audible, or a combination of tactile and audible. In one aspect the connector member may include a cantilevered member that is movable between neutral and expanded positions.

### BRIEF DESCRIPTION OF THE DRAWINGS

The invention will be more fully understood from the following detailed description taken in conjunction with the accompanying drawings, in which:

FIG. 1A is a side view of one embodiment of a connector member;

FIG. 1B is a disassembled view of the connector member of FIG. 1A;

FIG. 1C is a perspective view of the disassembled connector member of FIG. 1B;

FIG. 2 is a side view of another embodiment of a connector member;

FIG. 3A is a perspective view of a cross-connector including connector members as disclosed herein;

FIG. 3B is a top view of the cross-connector of FIG. 3A;

FIG. 3C is a partial, sectional view of one of the connector members on the cross-connector of FIG. 3A;

FIG. 3D is a side view of the cross-connector of FIG. 3A showing a spinal fixation element moving into a seating area of the connector member;

FIG. 3E is a side view of the cross-connector of FIG. 3D with two spinal fixation elements secured therein;

FIG. 4A is a side view of another embodiment of a cross-connector;

FIG. 4B is a partial, sectional view of one of the connector members on the cross-connector of FIG. 4A;

FIG. 4C is a side view of the cross-connector of FIG. 4A showing a spinal fixation element being seated within a seating area of one of the connector members;

FIG. 5A is a perspective view of yet another embodiment of a cross-connector disclosed herein that includes two connector members directly connected to one another;

FIG. 5B is a side view of the cross-connector of FIG. 5A;

FIG. 5C is a sectional view of the cross-connector of FIG. 5A;

FIG. 6A is a perspective view of still another embodiment of a cross-connector disclosed herein that includes an adjustable central portion;

FIG. 6B is a side view of the cross-connector of FIG. 6A;

FIG. 6C is a top view of the cross-connector of FIG. 6A; and

FIG. 6D is a partial, sectional view of the cross-connector of FIG. 6A.

### DETAILED DESCRIPTION OF THE INVENTION

Certain exemplary embodiments will now be described to provide an overall understanding of the principles of the structure, function, manufacture, and use of the devices and methods disclosed herein. One or more examples of these embodiments are illustrated in the accompanying drawings. Those skilled in the art will understand that the devices and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention.

Disclosed herein is a spinal fixation device including a mechanism that provides audible and/or tactile feedback when the connector is mated with a spinal fixation element. In one aspect, the spinal fixation device is a cross-connector adapted to mate with a spinal fixation element, such as a spinal rod, and which includes a seating area defined by opposed jaws. The cross-connector provides a feedback response when the spinal fixation element is properly seated between the opposed jaws. In one embodiment, a flexible cantilevered member provides the feedback. Further described herein is a method of connecting the cross-connector with a spinal fixation element. The method can include the steps of seating the spinal fixation element within the cross-connector and receiving a feedback response caused by movement of the cantilevered member between a neutral and an expanded position.

Although the connector and fixation devices are disclosed herein the context of a system used in spinal surgery, it is understood that the system can be adapted for use in a variety of other surgical procedures, including orthopedic procedures.

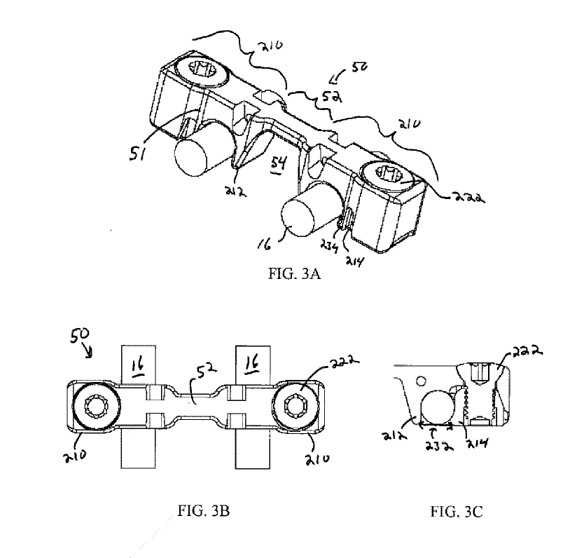

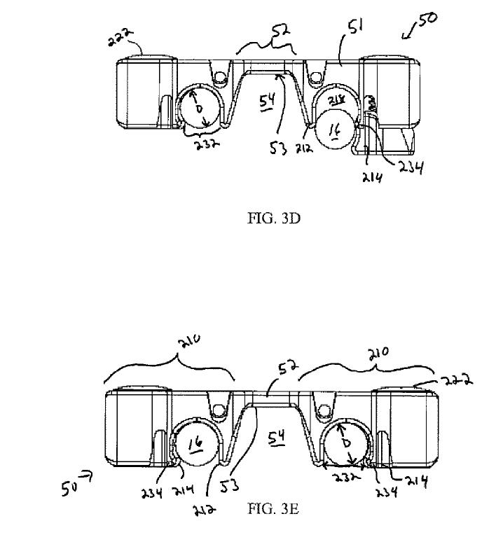

FIGS. 3A through 6D illustrate various embodiments of exemplary spinal devices which can generate a feedback response, as disclosed herein, when a spinal fixation element is properly seated. In each of the illustrated embodiments a cross-connector 50, 150, 250, 350 includes at least one connector member 10, 110, 210, 310, 410, 510. In one aspect, the connector members 10, 110, 210, 310, 410, 510 each have first 12, 112, 212, 312, 412, 512 and second 14, 114, 214, 314, 414, 514 opposed jaws. At least one of the first and second jaws is selectively movable between a first, open position in which the first and second jaws are positioned a distance apart from one another, and a second, closed position in which the first and second jaws are adapted to engage and maintain a spinal fixation element therebetween. In certain embodiments, the cross-connector 50, 150, 350 includes a generally elongate central portion 52, 152, 352 that can extend between an opposed pair of connector members to position the first and second connector members a desired distance apart from one another. The central portion 52, 152, 352 can have variety of configurations, for example, it can have a fixed length, which can vary depending on the intended use, or alternatively the central portion can have an adjustable length. The central portion can also be angularly and/or laterally adjustable to allow the connector members to be angularly positioned or offset with respect to a central axis of the central portion.

A person skilled in the art will appreciate that while each cross-connector 50, 150, 250, 350 is described herein as being adapted to engage a spinal fixation element, and in particular a cylindrical spinal fixation rod, that the cross-connectors can be configured to provide a feedback response upon seating a variety of spinal fixation elements, such as, anchors, cables, fixation plates, etc., having a variety of shapes. Moreover, the cross-connector can include only one connector member for engaging a spinal fixation element, and the opposed end of the cross-connector can be adapted for other uses. For example, the connector-free end of the cross-connector can be configured to be fixedly attached to a vertebra or another part of the anatomy.

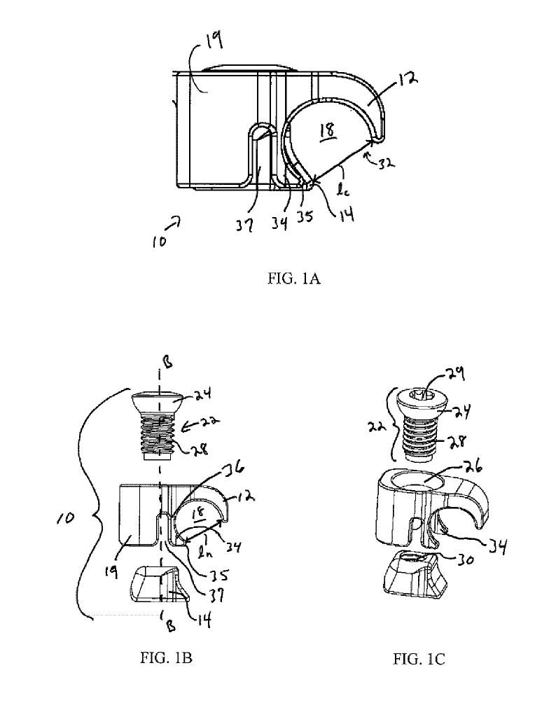

FIGS. 1A through 1C illustrate one embodiment of connector member 10 including a first jaw 12 and a second jaw 14 which are adapted to secure a spinal implant 16 (FIG. 3A). In one embodiment, connector member 10 includes a body 19 that can be unitary with at least one of jaws 12, 14. In the illustrated embodiment, jaw 12 is unitary with body 19 and is defined by a curved protruding portion of body 19. The inner surface of jaw 12 is generally of a shape such that it is complementary to the outer surface of a spinal fixation element which is to be seated therein. For example, inner surface of jaw 12 can be generally curved or c-shaped. Jaw 14 can be formed separately from body 19 and can also include an inner surface (e.g., curved and/or c-shaped) that is complementary to the fixation element 16 to be seated therein. As shown in FIGS. 1B and 1C, jaw 14 can be shaped and sized for positioning within an interior cavity of body 19 such that it is able to move generally or partially along an axis B-B between open and closed positions. In an alternative embodiment, jaws 12, 14 can both be unitary with body 19. In addition, while jaw 12 is shown as being unitary with body 19 and jaw 14 is shown as a separate component, an opposite arrangement can be utilized. One skilled in the art will appreciate that jaws 12, 14 can have a variety of alternative configurations, such as, for example, those disclosed in U.S. patent application Ser. No. 10/709,019, entitled “Spinal Cross-Connectors,” which is thereby incorporated by reference in its entirety.

In use, jaws 12, 14 can move between a closed position, in which the spinal fixation element is secured within a seating area 18 defined by jaws 12, 14, and an open position, in which the spinal fixation element can pass through a receiving area 32. In one aspect, jaw 14 can be moved between a first open position and a second closed position by way of a locking mechanism that is adapted to come into contact with the first and/or second jaws 12, 14 to move one or both jaws 12, 14 between open and closed positions. While virtually any locking mechanism can be used, in an exemplary embodiment the locking mechanism is a threaded member 22 having a head portion 24 and a threaded shank 28 as shown in FIGS. 1A through 1C. Threaded member 22 can be mated with body 19 such that the head portion 24 is adapted to sit within a recessed area 26 of body 19. Recessed area 26 can be shaped and sized to allow threaded member 22 to be rotated therein, while preventing head portion 24 from being removed axially therefrom. For example, recessed area 26 can include upper and lower diameters that are smaller than a maximum diameter of head portion 24, such that head portion 24 cannot pass through the upper and lower diameters of the recessed area.

To adjustably mate threaded member 22 to jaw 14, threaded member 22 can include a threaded shank 28 sized to engage with a threaded bore 30 within jaw 14. Movement of jaw 14 between an open and closed position can then be effected by rotation of threaded member 22. In one aspect, the connection between jaw 14 and threaded member 22 prevents jaw 14 from fully separating from connector member 10. For example, the lower portion of shank 28 can be swaged to prevent removal of jaw 14.

As shown in FIG. 1C, threaded member 22 can include a driver receiving element 29 positioned in head portion 24 for mating with a driver tool (not illustrated). In use, a male tool can mate with the driver receiving element 29, in the form of a recess, to move jaw 14 between open and closed positions. One skilled in the art will appreciate that while driver receiving element 29 is shown as a female recess, a variety of alternative driver receiving elements, such as, for example a male driver receiving element, could be used.

When jaws 12, 14 are in the closed position (FIG. 1A), spinal rod 16 cannot traverse receiving area 32 defined by an opening extending between jaws 12, 14. With jaws 12, 14 closed, the length l_{c} (i.e., the distance between jaws 12, 14) of receiving area 32 is less than the diameter of the spinal fixation element. When the jaws are in an open configuration, receiving area 32 is large enough to allow a spinal rod, for example, to pass between jaws 12, 14.

As spinal rod 16 passes through receiving area 32, a feedback response is preferably provided to the user. In one embodiment, a cantilevered member 34, capable of moving between a neutral position and an expanded position, is positioned within receiving area 32. In the neutral position the distance lₙ (FIG. 1B) between cantilevered member 34 and jaw 12 is less than the diameter of a spinal fixation element. As a user attempts to insert a spinal fixation element into seating area 18, the spinal fixation element will encounter the cantilevered member. Applying force sufficient to seat the spinal fixation element within seating area 18 will cause cantilevered member 34 to move into an expanded position, which will allow the spinal fixation element to pass through receiving area 32, and into seating area 18. Once the spinal fixation element has moved past receiving area 32, the cantilevered member will return to its original, neutral position which does not act on the spinal fixation element in such a way as to maintain it within seating area 18. The process of positioning a spinal fixation element within seating area 18, and the resulting deflection of cantilevered member 34, will cause a tactile and/or auditory feedback response alerting a user that the spinal fixation element is seated in seating area 18.

The dimension lₙ (FIG. 1B), as noted above, is less than a diameter of a spinal fixation element to be seated in seating area 18. The differential between dimension lₙ and the diameter of a spinal fixation element can vary depending upon many factors including the magnitude of feedback response desired, material properties, and other factors. In one embodiment, however, this differential is in the range of about 0.25 mm to about 0.75 mm.

As shown in FIGS. 1A through 1C, cantilevered member 34 can be positioned adjacent to jaw 14, and it includes a first end 35 that extends into receiving area 32. The length of the cantilevered member can vary, but it is generally in the range of about 3 mm to about 7 mm. On the side of cantilevered member 34 opposite seating area 18 is open area 37, which provides space in to which the cantilevered member may deflect away from jaw 12. One skilled in the art will appreciate that one or more cantilevered members can be utilized.

In one embodiment, cantilevered member 34 is defined by a portion of the cross-connector that extends from body 19 and which has a unitary construction therewith. The cantilevered member 34 can be connected to the body 19 a by living hinge 36, which is also referred to as an integrated hinge. In addition to connecting cantilevered member 34 to main body 19, the living hinge imparts flexibility to the cantilevered member 34 and allows it to deflect between neutral and expanded positions. The dimensions and properties of the living hinge can vary depending on numerous factors, including the degree of flexibility desired, the magnitude of the feedback response desired, the length of the cantilevered member the material from which the living hinge is formed, etc. In general, however, the thickness of the living hinge is in the range of about 0.5 mm to about 1 mm.

The living hinge can be formed of a variety of biocompatible materials that exhibit flexibility. In one embodiment, the living hinge is made from the same material as the cross-connector. Exemplary materials include metals, such as stainless steel and titanium; plastics, such as PEEK; and/or composites, such as fiber reinforced plastics.

As noted above, the amount of force required to move a spinal fixation element past the cantilevered member, and the force transmitted by the passage of the spinal fixation element through receiving area 32, will depend on a number of factors. These factors can include the dimensions of the cantilevered member 34 (length and cross-section), the cross-sectional dimensions of living hinge 36, the flexibility of the material from which living hinge 36 is formed, the spacing between the end of the cantilevered member 35 and jaw 12, and the diameter of the spinal fixation element. One skilled in the art will appreciate that these factors can be varied to provide a desired feedback response. In one aspect, these factors are adjusted to allow spinal fixation element 16 to pass through receiving area 32 without excessive resistance while causing a sufficiently noticeable tactile and/or audible feedback in response to the passage of the spinal fixation element.

Once the spinal fixation element has moved past receiving area 32 and cantilevered members 34, the spinal fixation element is seated within seating area 18. Jaw 14 can then be moved from the open position into the closed position, securing the spinal implant within seating area 18. In one embodiment, cantilevered members 34 are positioned such that when jaws 12, 14 are in the closed position a spinal implant secured therein is not held in the seated position by cantilevered member 34. That is, as shown in FIG. 1A, cantilevered member 34, is set back from seating area 18 and will not contact the spinal implant secured therein when the jaws are in the closed position. Conversely, when jaws 12, 14 are in the open position, cantilevered member 34 can be positioned within seating area 18, but it does not exert a force on the spinal fixation element sufficient to maintain it in the seated position.

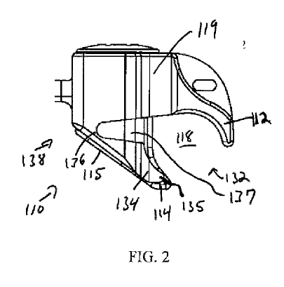

FIG. 2 illustrates another embodiment of the connector member disclosed herein. As shown, connector member 110 includes first and second jaws 112, 114, and cantilevered member 134. Jaw 112 is similar to jaw 12 described above and includes a curved or substantially c-shaped inner surface defining a portion of seating area 118. Jaw 114 can also have a surface defining seating area 118 for receiving a spinal fixation element, and as described above with respect to FIGS. 1A through 1C, jaw 114 can be positioned within body 119 of connector member 110. The shape of jaw 114 is similar to that of jaw 14, but jaw 114 includes a tapered portion 115, opposite the seating surface, that can provide additional clearance for anatomical features.

The cantilevered member 134 of connector member 110 operates in a manner similar to cantilevered member 34, but cantilevered member 134 is less vertically oriented than cantilevered member 34. As shown in FIG. 2, cantilevered member 134 extends from an inferior end 138 of body 119 toward seating area 118. Living hinge 136 is set back from seating area 118 and can, for example, be positioned at the inferior end of body 119. Connector member 110 can also include an open area 137 positioned between cantilevered member 134 and body 119. When cantilevered member 134 moves from the neutral position to the expanded position, open area 137 can increase in size.

Cantilevered member 134 is preferably shaped and sized such that end 135 is positioned in receiving area 132 when jaws 112, 114 are in the open position. As the spinal fixation element passes through receiving area 132, cantilevered member 134 flexes away from body 119. When jaws 112, 114 are closed, the receiving area can exclude end 135 of cantilevered member 134 such that the cantilevered member does not contact the spinal implant secured within receiving area 118.

FIGS. 3A through 3E illustrate one embodiment of a cross-connector 50 including a connector member 210 positioned at each end. Cross-connector 50 can include a generally elongate body 51 adapted for extending between two spinal fixation elements 16 (e.g., spinal rods). A central portion 52 extends between connector members 210 and defines an open region 54 which can receive anatomical features. In one embodiment, open region 54, defined by the bottom surface 53 of central portion 52 and the sides of connector members 210, is shaped and sized to receive or span of at least a portion of the spinous process.

As shown in FIGS. 3A through 3E, spinal rods 16 can be secured in each of connector members 210. Spinal rod 16 has a diameter D that can pass through receiving area 232 when the jaws are in the open position. However, the diameter of the spinal rod is larger than the distance between jaw 212 and cantilevered member 234. As shown in FIG. 3D, positioning spinal rods 16 in seating area 218 requires deflecting cantilevered member 234 into an expanded position to enable passage of rod 16 into seating area 218. Once spinal rod 16 is positioned within seating area 218, cantilevered member 234 springs back to its original, neutral position. The movement of cantilevered member 234 creates a feedback response which indicates to the user that spinal rod 16 is positioned within seating area 218. Jaws 212, 214 can then be brought together via locking mechanism 222, such that spinal rod 16 cannot pass through the reduced spacing of receiving area 232 as shown in FIG. 3E.

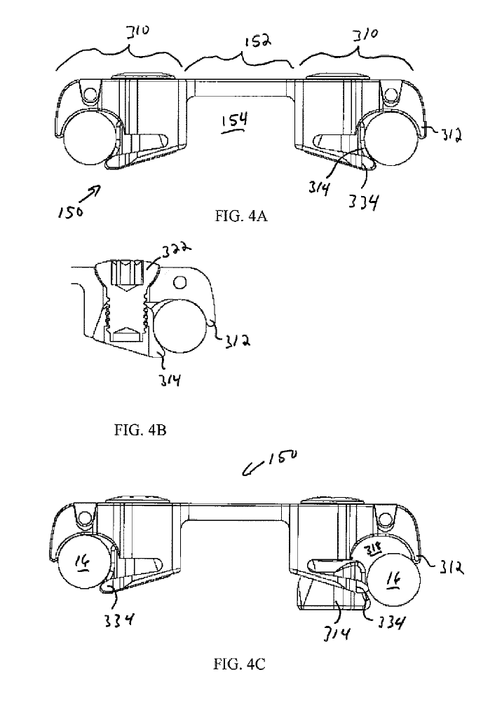

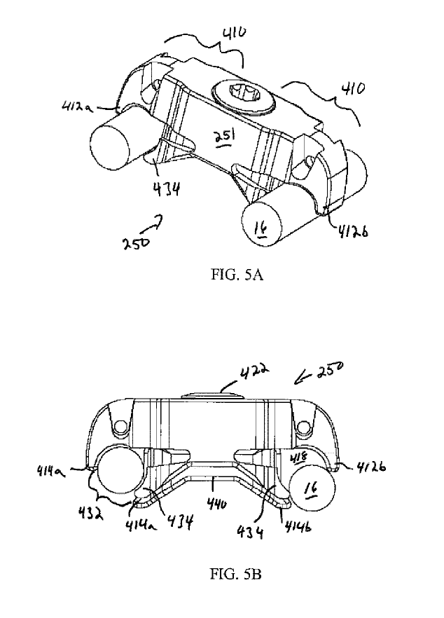

In one aspect, the connector members 210 of cross connector 50 are oriented such that seating areas 218 are positioned adjacent to open region 54 and locking mechanisms 222 are situated toward the opposed ends of the cross-connector body 51. Alternatively, the orientation of the connector members can be reversed. FIGS. 4A through 4C illustrate cross-connector 150 with seating areas 318 positioned adjacent to the opposed ends of the cross-connector with locking mechanism 322 positioned intermediate the opposed ends of the cross-connector and the seating areas 318. As a result, cross-connector 150 can mate with spinal rods 16 that are spaced apart by a greater distance. The central portion 152 of cross-connector 150 also affects the length of the cross-connector. One skilled in the art will appreciate that the central portion 152 can have a variety of lengths depending on the desired anatomical positioning and the expected location of the spinal devices to which the cross-connector will connect.

The open region 154 defined by the central portion and the connector members can also have a variety of shapes and sizes depending on the configuration of the central portion and connector members. For example, in FIG. 4A, central portion 152 is longer and open region 154 has a generally rectangular shape, as opposed to the generally trapezoidal shape of central portion 54 shown in FIG. 3A. One skilled in the art will appreciate that the open region can have a variety of shapes and sizes depending on the final placement of the cross-connector and the anatomical features which it will span.

In yet another embodiment, as shown in FIGS. 5A and 5B, a cross-connector 250 is provided that includes connector members 410 directly connected to one another as part of the same body. As illustrated, body 251 of cross-connector 250 does not require a central portion to connect connector members 410. Seating areas 418, into which spinal rods 16 are secured, are spaced apart only by the length of the connector members 410.

The cross-connector 250 includes jaws 412a, 412b formed from curved portions of body 251 and having a similar configuration to the jaws 112, 312 described above with respect to FIGS. 2 and 4A-4C. Jaws 414a, 414b, unlike jaws 14, 114, 214, 314 described above, are formed in unity on a single member 440 (FIGS. 5B and 5C). For example, jaws 414a, 414b can be positioned on opposite sides of member 440 and can include curved surfaces 441, 442 that define a portion of seating areas 418. Member 440 can have a size and shape adapted to fit in a cavity within body 251 and can move between an open position and a closed position.

As shown in FIG. 5C, a single locking mechanism 422 can be mated with member 440 such that jaws 414a, 414b are opened and closed in unison. In the open position, spinal fixation elements 16 can move through receiving area 432 and can sit within seating area 418. As the spinal fixation elements move through receiving area 432, they encounter cantilevered members 434 (FIG. 5B). Cantilevered members 434 can be formed in unity with body 251 of cross-connector 250 and positioned within receiving area 432 when jaws 412, 414 are in the open position. Cantilevered members 434 can create a feedback response as the spinal fixation members are moved into seating areas 418. Locking mechanism 422 can then be actuated to move member 440 into a locked position, thereby securing spinal fixation elements 16 within seating areas 418.

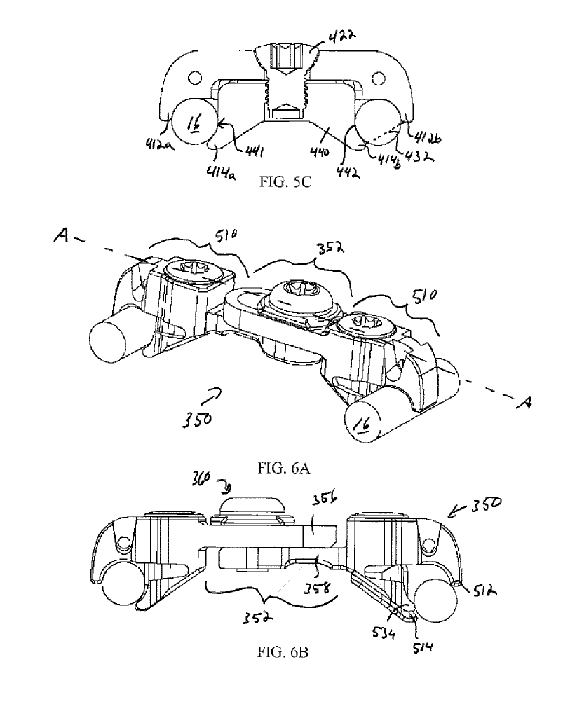

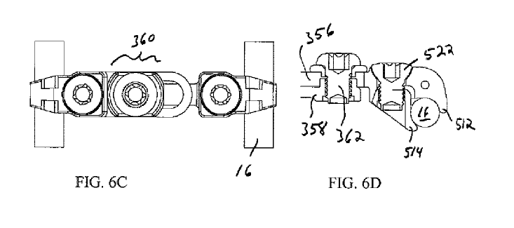

As mentioned above, the central portion of the cross-connectors 50, 150, 350 can incorporate a variety of features including lateral and angular adjustability. FIGS. 6A through 6D illustrate one embodiment of cross-connector 350 having central portion 352 that includes first and second generally elongate transverse members 356, 358 that extend along a longitudinal axis A of the cross-connector. The transverse members 356, 358 are slidably mated to one another by a central locking mechanism 360, which includes a threaded fixation element 362 that extends through traverse member 356 to a threaded bore within traverse member 358. When unlocked, central locking mechanism 360 allows slidable and/or angular movement of the transverse members 356, 358 and when locked the central locking mechanism is effective to fix traverse members 356, 358 in a position with respect to one another by actuating fixation element 362. While locking mechanism 360 provides central portion 352 with an adjustable length, one skilled in the art will appreciate that a variety of locking mechanisms that allow adjustments to the angle and/or length of central portion 352 could be substituted for locking mechanism 360. Exemplary locking mechanisms are described in U.S. patent application Ser. No. 10/709,019, entitled “Spinal Cross-Connectors,” which is referenced above and incorporated herein by reference.

A method of using the connector members is also provided. In one embodiment, spinal rods are implanted on either side of a vertebral column. A cross-connector having at least one connector member extending therefrom is then connected to an implanted spinal rod. The steps of connecting the cross-connector can include seating a spinal rod in the seating area between first and second jaws. As the spinal rod passes through the receiving area the spinal rod engages a cantilevered member causing the cantilevered member to move from a neutral position to an expanded position. As the spinal rod passes the cantilevered member, a feedback response is transmitted to a user. In one embodiment, a tactile feedback response is transmitted through the cross-connector and/or inserter tools to a user. Alternatively, or additionally, a user may hear an audible feedback response.

Once the spinal rod is seated within the seating area, the jaws can be brought together to fix the spinal rod with respect to the cross-connector. In one embodiment, a tool is mated with locking mechanism and a threaded member is rotated to bring the jaws into a spinal rod securing position.

One skilled in the art will appreciate further features and advantages of the invention based on the above-described embodiments. Accordingly, the invention is not to be limited by what has been particularly shown and described, except as indicated by the appended claims. All publications and references cited herein are expressly incorporated herein by reference in their entirety.

What is claimed is:

1. An implantable spinal cross-connector, comprising:
at least one connector member having a main body and first and second cooperating jaws defining a seating area and adapted to receive a spinal fixation element therebetween; and
a cantilevered member extending into the seating area and being movable between neutral and expanded positions in response to insertion of a spinal fixation element into the seating area, the cantilevered member providing a feedback response upon movement between the neutral and expanded positions in response to insertion of a spinal fixation element into the seating area.

2. The cross-connector of claim 1, wherein the first jaw is unitary with the main body.

3. The cross-connector of claim 1, wherein the second jaw is mated to the main body.

4. The cross-connector of claim 3, wherein the second jaw is adapted to move between a first, open position and a second position effective to secure a spinal fixation element in the seating area.

5. The cross-connector of claim 1, further including a locking mechanism effective to move at least one of the first and second jaws between an open position and a closed position in which the jaws are effective to secure a spinal fixation element therebetween.

6. The cross-connector of claim 1, wherein the feedback response is selected from the group consisting of audible, tactile, and combinations thereof.

7. The cross-connector of claim 1, wherein the cantilevered member is connected to the main body by a living hinge.

8. The cross-connector of claim 1, wherein the cantilevered member is spaced from one of the first and second jaws to define a receiving space, the receiving space having a dimension that is less than a diameter of a fixation element.

9. An implantable spinal cross-connector, comprising:
a central portion with at least one connector member formed on a terminal end thereof, the at least one connector member having first and second opposed jaws, at least one of the jaws being selectively movable between a first, open position wherein the first and second jaws are positioned a first distance apart from one another, and a second, closed position wherein the first and second jaws are spaced to engage a spinal fixation element therebetween; and
a cantilevered member positioned between the first and second jaws to define a receiving space between the cantilevered member and one of the jaws having a dimension less than the first distance.

10. The cross-connector of claim 9, further including a locking mechanism effective to move at least one of the first and second jaws between the first position and the second position.

11. The cross-connector of claim 9, wherein the cantilevered member provides a feedback response when a spinal implant is moved through the receiving space between the first and second jaws.

12. The cross-connector of claim 11, wherein the feedback response is selected from the group consisting of audible, tactile, and combinations thereof.

13. The cross-connector of claim 9, wherein the cantilevered member is connected to the main body by a living hinge.

14. The cross-connector of claim 9, wherein the central portion has two terminal ends and a connector member is disposed on each terminal end.

15. An implantable system, comprising:
a spinal fixation element including at least a portion thereof having a substantially cylindrical shape; and
a cross-connector including a main body with at with at least a first jaw, the first jaw defining at least a portion of a seating area able to receive a spinal fixation element; and
a cantilevered member extending from the main body, the cantilevered member being opposed to the first jaw and spaced apart from the first jaw by a distance less than a diameter of the spinal fixation element, wherein the cantilevered member is able to deflect to enable the spinal implant to be inserted into the seating area and to return to its original position once the spinal fixation element is disposed in the seating area.

16. The system of claim 15, wherein the cross-connector includes a second jaw opposed to the first jaw, and positioned such that the cantilevered member is between the first and second jaws when the jaws are in an open position.

17. The system of claim 16, wherein at least one of the first and second jaws is movable between the open position effective to receive the spinal implant and a closed position effective to securely seat the spinal fixation element.

18. A method of connecting to a spinal implant, comprising
providing at least one connector member having a main body and first and second cooperating jaws defining a seating area and adapted to receive a spinal fixation element therebetween, and;
positioning a spinal implant within in the seating area such that a feedback response is generated in response to insertion of the spinal fixation element into the seating area.

19. The cross-connector of claim 18, wherein the feedback response is selected from the group consisting of audible, tactile, and combinations thereof.

20. The cross-connector of claim 18, wherein the connector member further includes a cantilevered member movable between neutral and expanded positions.
